(19) United States
(12) Patent Application Publication
(10) Pub. No.: US 2023/0149225 A1
(43) Pub. Date: May 18, 2023

(54) PADDED UNDERGARMENT

(71) Applicant: Velicia Kelly-Banks, Red Wing, MN (US)

(72) Inventor: Velicia Kelly-Banks, Red Wing, MN (US)

(21) Appl. No.: 17/529,641

(22) Filed: Nov. 18, 2021

(51) Int. Cl. A61F 13/49; A61F 13/511; A61F 13/513

(52) U.S. Cl. CPC A61F 13/49017; A61F 13/5116; A61F 13/51305; A61F 2013/49088

(57) ABSTRACT

A padded undergarment, including a main body to be worn around a pelvis of a user, and an absorbent pad disposed within at least a portion of the main body to absorb a fluid discharge thereon.

### BACKGROUND

### 1. Field

The present general inventive concept relates generally to an undergarment, and particularly, to a padded undergarment.

### 2. Description of the Related Art

An onset of menstruation (a.k.a., a menstrual period) is often a confusing and uncomfortable experience for a young girl. Moreover, modern designs of menstrual pads can take time and be inconvenient and/or uncomfortable to use. Also, menstrual pads often have sizes that may be too big and bulky for young girls with petite frames.

Generally, conventional undergarments for women are not equipped to provide protection from discharge released during the menstrual period. The discharge of fluids during the menstrual period can be sudden, which can leave a mess and be embarrassing for the young girl.

Therefore, there is a need for a padded undergarment that prevents leaking of fluid without requiring a separate menstrual pad.

### SUMMARY

The present general inventive concept provides a padded undergarment.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a padded undergarment, including a main body to be worn around a pelvis of a user, and an absorbent pad disposed within at least a portion of the main body to absorb a fluid discharge thereon.

The main body may include a plurality of leg receiving apertures disposed on at least a portion of a bottom of the main body, each of the plurality of leg receiving apertures to receive a limb of the user therethrough, and a crotch section disposed on at least a portion of the bottom portion of the main body to at least partially cover a crotch of the user.

The absorbent pad may be disposed within at least a portion of the crotch section.

The absorbent pad may include a front section to disperse the fluid discharge away from a top surface of the front section toward a bottom surface of the front section, and a rear section disposed on at least a portion of the front section to disperse the fluid discharge away from a top surface of the rear section toward a bottom surface of the rear section.

The padded undergarment may further include an elastic unit disposed within at least a portion of the absorbent pad to at least partially deform in response to an application of force thereto, such that the elastic unit adjusts a shape of the absorbent pad.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1A illustrates a front perspective view of a padded undergarment, according to an exemplary embodiment of the present general inventive concept;

FIG. 1B illustrates a rear perspective view of the padded undergarment, according to an exemplary embodiment of the present general inventive concept;

FIG. 2A illustrates a front perspective view of a padded undergarment, according to another exemplary embodiment of the present general inventive concept; and

FIG. 2B illustrates a rear perspective view of the padded undergarment, according to another exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Padded Undergarment 100
Main Body 110
Leg Receiving Apertures 111
Crotch Section 112
Absorbent Pad 120
Front Section 121
Rear Section 122
Elastic Unit 130
Padded Undergarment 200
Main Body 210
Leg Receiving Apertures 211
Crotch Section 212
Absorbent Pad 220
Front Section 221
Rear Section 222
Elastic Unit 230

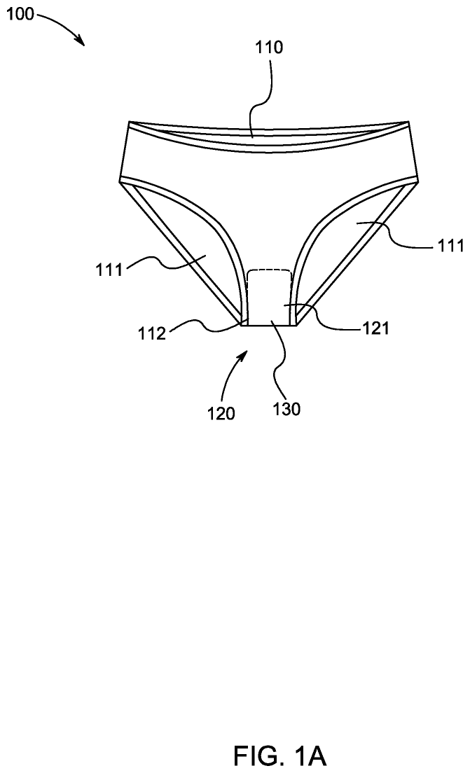

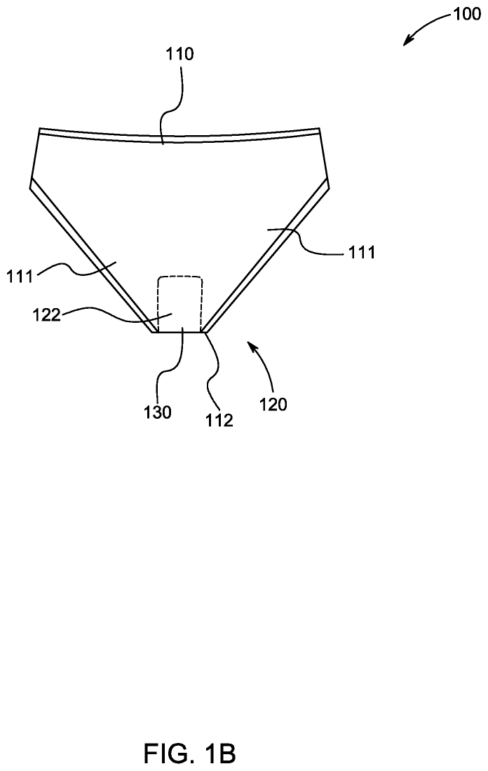

FIG. 1A illustrates a front perspective view of a padded undergarment 100, according to an exemplary embodiment of the present general inventive concept.

FIG. 1B illustrates a rear perspective view of the padded undergarment 100, according to an exemplary embodiment of the present general inventive concept.

The padded undergarment 100 may be constructed from at least one of cloth, spandex, elastane, plastic, and rubber, etc., but is not limited thereto. Also, the padded undergarment 100 may be constructed of hypoallergenic material to prevent skin irritation, and biodegradable.

The padded undergarment 100 may include a main body 110, an absorbent pad 120, and an elastic unit 130, but is not limited thereto.

Referring to FIGS. 1A and 1B, the main body 110 is illustrated to be a panty. However, the main body 110 may be a brief, a thong, a G-string, or any type of undergarment known to one of ordinary skill in the art, but is not limited thereto.

The main body 110 may be removably connected to at least a portion of a pelvis of a user. In other words, the main body 110 may be worn around the pelvis of the user to at least partially cover the pelvis of the user, such as genitalia and/or buttocks of the user.

The main body 110 may include a plurality of leg receiving apertures 111 and a crotch section 112, but is not limited thereto.

The plurality of leg receiving apertures 111 may be disposed on at least a portion of a bottom of the main body 110. Moreover, each of the plurality of leg receiving apertures 111 may be distanced from each other. Each of the plurality of leg receiving apertures 111 may receive a limb (e.g., a leg) of the user therethrough.

The crotch section 112 may be disposed on at least a portion of the bottom portion of the main body 110 between the plurality leg receiving apertures 111. The crotch section 112 may at least partially cover a crotch of the user. Referring again to FIGS. 1A and 1B, the crotch section 112 may extend from a front portion of the main body to a rear portion of the main body 110, such as between the genitalia and/or the buttocks of the user.

The absorbent pad 120 may include a front section 121 and a rear section 122, but is not limited thereto.

The front section 121 of the absorbent pad 120 may be disposed within at least a portion of a first side of the main body 110, such that the front section 121 may be disposed between fabric layers of the main body 110. The front section 121 may receive and/or absorb a fluid discharge thereon. Additionally, the front section 121 may disperse the fluid discharge away from a top surface of the front section 121 toward a bottom surface of the front section 121, such that the fluid discharge may not remain in contact with a crotch of the user. As such, the top surface of the front section 121 may remain dry. Also, the front section 121 may include a plurality of layers to facilitate dispersing the fluid discharge.

The rear section 122 of the absorbent pad 120 may be disposed within at least a portion of a second side of the main body 110 different from the first side, and on at least a portion of the first section 121, such that the rear section 122 may be disposed between the fabric layers of the main body 110. The rear section 122 may receive and/or absorb the fluid discharge thereon. Additionally, the rear section 122 may disperse the fluid discharge away from a top surface of the rear section 122 toward a bottom surface of the rear section 122, such that the fluid discharge may not remain in contact with the crotch and/or the buttocks of the user. As such, the top surface of the rear section 122 may remain dry. Also, the rear section 122 may include a plurality of layers to facilitate dispersing the fluid discharge.

Furthermore, the absorbent pad 120 may be disposed within at least a portion of the crotch section 112.

The elastic unit 130 may include a rubber, polydimethylsiloxane (i.e., putty), and a wire, but is not limited thereto.

The elastic unit 130 may be disposed on and/or within at least a portion of the absorbent pad 120. The elastic unit 130 may at least partially deform (i.e., bend, stretch, expand) in response to an application of force (e.g., pushing, pulling) thereto. More specifically, the elastic unit 130 may adjust a shape of the absorbent pad 120 in response to the application of force, such that the absorbent pad 120 may remain in at least one shape in absence of the application of force. As such, the elastic unit 130 may adjust the shape of the absorbent pad 120 based on a preference of the user. Accordingly, the absorbent pad 120 may be adjusted to provide comfort to the user based on changing a fit of the absorbent pad 120.

Therefore, the padded undergarment 100 may prevent leaks of the fluid discharge received thereon. Also, the padded undergarment 100 may avoid difficulty of having to connect an external pad to the padded undergarment 100.

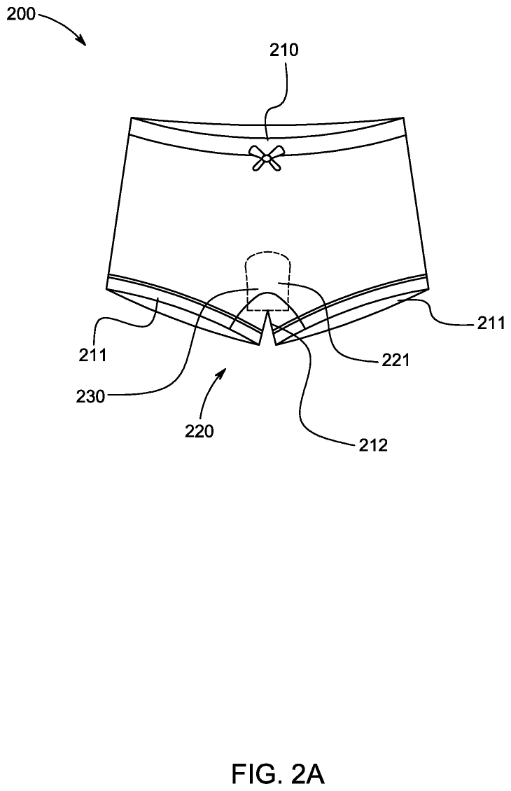

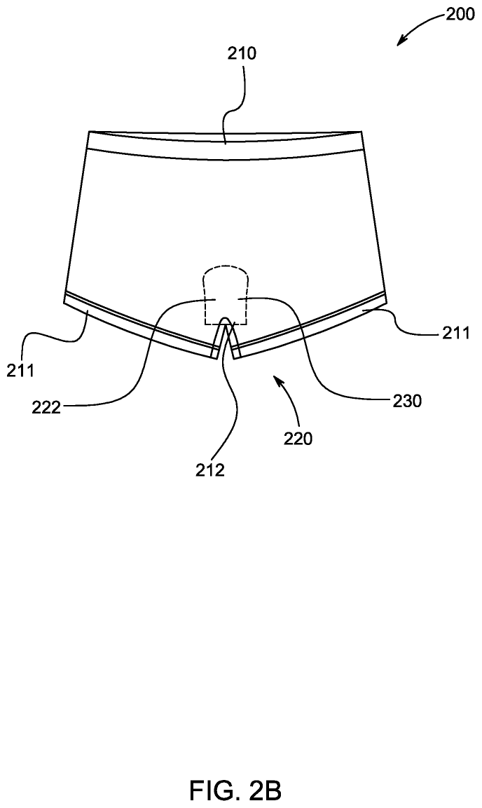

FIG. 2A illustrates a front perspective view of a padded undergarment 200, according to another exemplary embodiment of the present general inventive concept.

FIG. 2B illustrates a rear perspective view of the padded undergarment 200, according to another exemplary embodiment of the present general inventive concept.

The padded undergarment 200 may be constructed from at least one of cloth, spandex, elastane, plastic, and rubber, etc., but is not limited thereto. Also, the padded undergarment 200 may be constructed of hypoallergenic material to prevent skin irritation, and biodegradable.

The padded undergarment 200 may include a main body 210, an absorbent pad 220, and an elastic unit 230, but is not limited thereto.

Referring to FIGS. 2A and 2B, the main body 210 is illustrated to be a boxer. However, the main body 210 may be a brief, a boxer brief, a thong, a G-string, or any type of undergarment known to one of ordinary skill in the art, but is not limited thereto.

The main body 210 may be removably connected to at least a portion of a pelvis of a user. In other words, the main body 210 may be worn around the pelvis of the user to at least partially cover the pelvis of the user, such as genitalia and/or buttocks of the user.

The main body 210 may include a plurality of leg receiving apertures 211 and a crotch section 212, but is not limited thereto.

The plurality of leg receiving apertures 211 may be disposed on at least a portion of a bottom of the main body 210. Moreover, each of the plurality of leg receiving apertures 211 may be distanced from each other. Each of the plurality of leg receiving apertures 211 may receive a limb (e.g., a leg) of the user therethrough.

The crotch section 212 may be disposed on at least a portion of the bottom portion of the main body 210 between the plurality leg receiving apertures 211. The crotch section 212 may at least partially cover a crotch of the user. Referring again to FIGS. 2A and 2B, the crotch section 212 may extend from a front portion of the main body to a rear portion of the main body 210, such as between the genitalia and/or the buttocks of the user.

The absorbent pad 220 may include a front section 221 and a rear section 222, but is not limited thereto.

The front section 221 of the absorbent pad 220 may be disposed within at least a portion of a first side of the main body 210, such that the front section 221 may be disposed between fabric layers of the main body 110. The front section 221 may receive and/or absorb a fluid discharge thereon. Additionally, the front section 221 may disperse the fluid discharge away from a top surface of the front section 221 toward a bottom surface of the front section 221, such that the fluid discharge may not remain in contact with a crotch of the user. As such, the top surface of the front section 221 may remain dry. Also, the front section 221 may include a plurality of layers to facilitate dispersing the fluid discharge.

The rear section 222 of the absorbent pad 220 may be disposed within at least a portion of a second side of the main body 210 different from the first side, and on at least a portion of the first section 221, such that the rear section 222 may be disposed between the fabric layers of the main body 210. The rear section 222 may receive and/or absorb the fluid discharge thereon. Additionally, the rear section 222 may disperse the fluid discharge away from a top surface of the rear section 222 toward a bottom surface of the rear section 222, such that the fluid discharge may not remain in contact with the crotch and/or the buttocks of the user. As such, the top surface of the rear section 222 may remain dry. Also, the rear section 222 may include a plurality of layers to facilitate dispersing the fluid discharge.

Furthermore, the absorbent pad 220 may be disposed within at least a portion of the crotch section 212.

The elastic unit 230 may include a rubber, polydimethylsiloxane (i.e., putty), and a wire, but is not limited thereto.

The elastic unit 230 may be disposed on and/or within at least a portion of the absorbent pad 220. The elastic unit 230 may at least partially deform (i.e., bend, stretch, expand) in response to an application of force (e.g., pushing, pulling) thereto. More specifically, the elastic unit 230 may adjust a shape of the absorbent pad 220 in response to the application of force, such that the absorbent pad 220 may remain in at least one shape in absence of the application of force. As such, the elastic unit 230 may adjust the shape of the absorbent pad 220 based on a preference of the user. Accordingly, the absorbent pad 220 may be adjusted to provide comfort to the user based on changing a fit of the absorbent pad 220.

Therefore, the padded undergarment 200 may prevent leaks of the fluid discharge received thereon. Also, the padded undergarment 200 may avoid difficulty of having to connect an external pad to the padded undergarment 200.

The present general inventive concept may include a padded undergarment 100, including a main body 110 to be worn around a pelvis of a user, and an absorbent pad 120 disposed within at least a portion of the main body 110 to absorb a fluid discharge thereon.

The main body 110 may include a plurality of leg receiving apertures 111 disposed on at least a portion of a bottom of the main body 110, each of the plurality of leg receiving apertures 111 to receive a limb of the user therethrough, and a crotch section 112 disposed on at least a portion of the bottom portion of the main body 110 to at least partially cover a crotch of the user.

The absorbent pad 120 may be disposed within at least a portion of the crotch section 112.

The absorbent pad 120 may include a front section 121 to disperse the fluid discharge away from a top surface of the front section 121 toward a bottom surface of the front section 121, and a rear section 122 disposed on at least a portion of the front section 121 to disperse the fluid discharge away from a top surface of the rear section 122 toward a bottom surface of the rear section 122.

The padded undergarment 100 may further include an elastic unit 130 disposed within at least a portion of the absorbent pad 120 to at least partially deform in response to an application of force thereto, such that the elastic unit 130 adjusts a shape of the absorbent pad 120.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A padded undergarment, comprising:
a main body to be worn around a pelvis of a user; and
an absorbent pad disposed within at least a portion of the main body to absorb a fluid discharge thereon.

2. The padded undergarment of claim 1, wherein the main body comprises:
a plurality of leg receiving apertures disposed on at least a portion of a bottom of the main body, each of the plurality of leg receiving apertures to receive a limb of the user therethrough; and
a crotch section disposed on at least a portion of the bottom portion of the main body to at least partially cover a crotch of the user.

3. The padded undergarment of claim 2, wherein the absorbent pad is disposed within at least a portion of the crotch section.

4. The padded undergarment of claim 1, wherein the absorbent pad comprises:
a front section to disperse the fluid discharge away from a top surface of the front section toward a bottom surface of the front section; and
a rear section disposed on at least a portion of the front section to disperse the fluid discharge away from a top surface of the rear section toward a bottom surface of the rear section.

5. The padded undergarment of claim 1, further comprising:
an elastic unit disposed within at least a portion of the absorbent pad to at least partially deform in response to an application of force thereto, such that the elastic unit adjusts a shape of the absorbent pad.
